(19) United States
(12) Patent Application Publication
(10) Pub. No.: US 2023/0200750 A1
(43) Pub. Date: Jun. 29, 2023

(54) INFORMATION PROCESSING METHOD, INFORMATION PROCESSING DEVICE, AND NON-TRANSITORY COMPUTER READABLE RECORDING MEDIUM STORING INFORMATION PROCESSING PROGRAM

(71) Applicant: Panasonic Intellectual Property Corporation of America, Torrance, CA (US)

(72) Inventors: Toshiaki TANAKA, Hyogo (JP); Kana MIYATA, Osaka (JP); Yuki MINODA, Hyogo (JP); Ken OHMAE, Tokyo (JP); Yushi NAKAMURA, Osaka (JP); Koh UEHARA, Osaka (JP)

(73) Assignee: Panasonic Intellectual Property Corporation of America, Torrance, CA (US)

(21) Appl. No.: 18/112,909

(22) Filed: Feb. 22, 2023

Related U.S. Application Data
(63) Continuation of application No. PCT/JP2021/030866, filed on Aug. 23, 2021.
(60) Provisional application No. 63/072,318, filed on Aug. 31, 2020.

(30) Foreign Application Priority Data
Aug. 4, 2021 (JP) 2021-128550

(51) Int. Cl. A61B 5/00; A61B 5/01; A61B 5/117

(52) U.S. Cl. CPC A61B 5/746; A61B 5/01; A61B 5/117

(57) ABSTRACT

A body temperature management server executes: acquiring body temperature information indicating a body temperature of a user and associated with a user ID specifying the user; acquiring user information about the user associated with the user ID; acquiring an alert criterion in accordance with the user information; determining whether the body temperature information satisfies the alert criterion; generating alert information for issuing an alert about a health state of the user in association with the user ID when the body temperature information is determined to satisfy the alert criterion; and outputting the alert information.

### TECHNICAL FIELD

This disclosure relates to a technology of acquiring body temperature information indicating a body temperature of a user, and presenting the acquired body temperature information to another user.

### BACKGROUND ART

For instance, Patent Literature 1 discloses that a database server retrieves, upon receipt of: an employee number of an employee who is individually identified; a body temperature measured value; and time information from an information acquisition device, a corresponding employee test file from an external storage device on the basis of the employee number and the time information, and stores the time information and the body temperature measured value received from the information acquisition device in a field of the retrieved employee test file. Patent Literature 1 further discloses that the database server causes a display to display a department sheet associating a name, a clock-in time, and a body temperature measured value of an employee with one another.

However, the conventional technology fails to consider presentation of alert information in accordance with an environment around a user or a situation of the user, and thus needs further improvement.

### CITATION LIST

### Patent Literature

Patent Literature 1: Japanese Unexamined Patent Publication No. 2006-293766

### SUMMARY OF INVENTION

This disclosure has been achieved to solve the drawbacks described above, and has an object of providing a technology for enabling presentation of alert information in accordance with an environment around a user or a situation of the user.

An information processing method according to this disclosure, by a computer, includes: acquiring body temperature information indicating a body temperature of a user and associated with a user Ill specifying the user; acquiring user information about the user associated with the user ID; acquiring an alert criterion in accordance with the user information from an alert criteria database; determining whether the body temperature information satisfies the alert criterion; generating alert information for issuing an alert about a health state in association with the user ID when the body temperature information is determined to satisfy the alert criterion; and outputting the alert information.

This disclosure achieves presentation of alert information in accordance with an environment around a user or a situation of the user.

### BRIEF DESCRIPTION OF DRAWINGS

FIG. 1 is a diagram showing an example of a configuration of a body temperature management system in a first embodiment of the disclosure.

FIG. 2 is a diagram showing an example of a configuration of a body temperature management server in the first embodiment of the disclosure.

FIG. 3 shows an example of an alert criteria DB in the first embodiment.

FIG. 4 is a flowchart explaining notification of information by the body temperature management server in the first embodiment of the disclosure.

FIG. 5 shows an example of a body temperature presentation screen image displayed on a second user terminal in the first embodiment.

FIG. 6 shows an example of an alert presentation screen image displayed on the second user terminal in the first embodiment.

FIG. 7 is a diagram showing an example of a body temperature management server in a second embodiment of the disclosure.

FIG. 8 shows an example of an alert criteria DB in the second embodiment.

FIG. 9 is a flowchart explaining notification of information by the body temperature management server in the second embodiment of the disclosure.

FIG. 10 is a diagram showing an example of a body temperature management server in a third embodiment of the disclosure.

FIG. 11 shows an example of user information in the third embodiment.

FIG. 12 shows an example of an alert criteria DB in the third embodiment.

FIG. 13 shows an example of an alert destination DB in the third embodiment.

FIG. 14 is a first flowchart explaining notification of information by the body temperature management server in the third embodiment of the disclosure.

FIG. 15 is a second flowchart explaining the notification of information by the body temperature management server in the third embodiment of the disclosure.

FIG. 16 shows an example of an alert presentation screen image displayed on a second user terminal in the third embodiment.

FIG. 17 shows another example of the alert presentation screen image displayed on the second user terminal in the third embodiment of the disclosure.

### DESCRIPTION OF EMBODIMENTS

Knowledge forming the basis of the present disclosure

In the aforementioned conventional technology, a display displays a department sheet associating a name, a clock-in time, and a body temperature measured value of an employee with one another thereon. For instance, a manager of a certain department can check body temperatures of respective employees in the department at their clock-in time by seeing the department sheet displayed on the display. However, only the presentation of the body temperatures of the employees makes it difficult for the manager to determine which employee needs attention.

Therefore, alert information is preferably presented together with a body temperature to the manager. However, a criterion for presentation of the alert information varies in accordance with an environment around each user or a situation thereof. For instance, an alert level differs between a case where an employee living in a region with a small number of virus infected persons has a fever and a case where an employee living in a region having a large number of virus infected persons has a fever. Specifically, the alert level for the employee living in the region having the large number of virus infected persons is higher than the alert level for the employee living in the region having the small number of virus infected persons. In this respect, the conventional technology fails to consider presentation of alert information in accordance with an environment around a user or a situation thereof.

To solve the above-described drawbacks, an information processing method according to one aspect of this disclosure, by a computer, includes: acquiring body temperature information indicating a body temperature of a user and associated with a user ID specifying the user; acquiring user information about the user associated with the user ID; acquiring an alert criterion in accordance with the user information from an alert criteria database; determining whether the body temperature information satisfies the alert criterion; generating alert information for issuing an alert about a health state in association with the user ID when the body temperature information is determined to satisfy the alert criterion; and outputting the alert information.

According to this configuration, an alert criterion is acquired in accordance with the user information from the alert criteria data base, and alert information for issuing an alert about a health state of the user is output when body temperature information is determined to satisfy the alert criterion. This achieves presentation of alert information in accordance with an environment around the user or a situation of the user.

The information processing method may further include acquiring dangerous level information indicating a dangerous level per region in accordance with a virus spread situation. The user information may include residence information indicating a residence of the user. The alert criteria database may associate a plurality of dangerous levels with alert criteria for the body temperature. In the acquiring of the alert criterion, a specific dangerous level for the residence of the user may be specified on the basis of the dangerous level information, and the alert criterion associated with the specified dangerous level may be acquired from the alert criteria database.

According to this configuration, a dangerous level for the residence of the user is specified on the basis of the dangerous level information indicating the dangerous level per region in accordance with the virus spread situation, and the alert criterion associated with the specified dangerous level is acquired from the alert criteria data base. Therefore, the alert information is presentable in accordance with the virus spread situation in the residence of the user.

In the information processing method, the user information may include the number of contact persons with the user on a previous day, and the alert criteria database may associate the number of contact persons with the alert criterion for the body temperature. In the acquiring of the alert criterion, the alert criterion associated with the acquired number of contact persons with the user on the previous day may be acquired from the alert criteria database.

According to this configuration, the alert criterion associated with the number of contact persons with the user on the previous day is acquired from the alert criteria data base. Therefore, the alert information is presentable in accordance with the number of contact persons with the user on the previous day.

The information processing method may further include outputting the body temperature information when the body temperature information is determined to dissatisfy the alert criterion.

This configuration achieves presentation of the body temperature information without presenting alert information when the body temperature information is determined to dissatisfy the alert criterion.

In the information processing method, the alert criteria database may further associate the alert criterion with an alert level. The information processing method may further include: specifying the alert level associated with the alert criterion when the body temperature information is determined to satisfy the alert criterion; and specifying, with reference to an alert destination database associating the alert level with an alert destination, the alert destination associated with the specified alert level. In the outputting of the alert information, the alert information may be output to the specified alert destination.

According to this configuration, when the body temperature information is determined to satisfy the alert criterion, an alert level associated with the alert criterion is specified, an alert destination associated with the specified alert level is specified, and alert information is output to the specified alert destination. Hence, the alert destination is changeable in accordance with the alert criterion. For instance, an amount of alert information to be presented to a person at an alert destination is reducible by changing an alert level associated with the alert destination in accordance with the number of users managed by the person at the alert destination. This succeeds in preventing an increase in an amount of complicated work of confirming the alert information.

In the information processing method, the alert criteria database may further associate the alert criterion with an alert level. The information processing method may further include specifying the alert level associated with the alert criterion when the body temperature information is determined to satisfy the alert criterion. In the generating of the alert information, the alert information having a display manner in accordance with the specified alert level may be generated.

According to this configuration, when the body temperature information is determined to satisfy the alert criterion, an alert level associated with the alert criterion is specified, and alert information having a display manner in accordance with the specified alert level is generated. Hence, the display manner for the alert information is changeable in accordance with the alert level. For instance, the display manner for the alert information is changeable in accordance with a virus spread situation in a region where the user lives, and thus easy visual confirmation of the user who needs further attention is attained.

In the information processing method, the user information may include at least one of: health condition information indicating a health condition of the user; a past body temperature history of the user: residence information indicating a residence of the user; underlying disease information about an underlying disease of the user; age information indicating the age of the user; and contact person information indicating the number of contact persons with the user on a previous day. The alert criteria database may include a first alert criterion for the body temperature, and a second alert criterion for at least one of: a health condition; a fever continuous day number indicating the number of days when the body temperature continuously shows a predetermined temperature or higher; a dangerous level per region in accordance with a virus spread situation; the underlying disease; the age; and the number of contact persons on the previous day. In the determining whether to satisfy the alert criterion, the body temperature information may be determined whether to satisfy the first alert criterion, and at least one of the health condition information, the fever continuous day number, a dangerous level in the residence, the underlying disease information, the age information, and the contact person information may be determined whether to satisfy the second alert criterion. The alert information may be generated when the body temperature information is determined to satisfy the first alert criterion, and the at least one of the health condition information, the fever continuous day number, the dangerous level in the residence, the underlying disease information, the age information, and the contact person information is determined to satisfy the second alert criterion.

According to this configuration, alert information is generated when the body temperature information is determined to satisfy the first alert criterion, and at least one of the health condition information, the fever continuous day number, the dangerous level in the residence, the underlying disease information, the age information, and the contact person information is determined to satisfy the second alert criterion.

Therefore, the alert information is output in consideration of at least one of the health condition information, the fever continuous day number, the dangerous level in the residence, the underlying disease information, the age information, and the contact person information, in addition to the body temperature information. This achieves presentation of alert information in accordance with a further exact environment around the user or a further exact situation of the user.

Moreover, the disclosure can be realized as: the information processing method executing the above-described distinctive ways; and an information processing device including each distinctive feature corresponding to the distinctive ways executed by the information processing method. Additionally, the disclosure can be realized by a computer program causing a computer to execute the distinctive ways included in the information processing method. From these perspectives, the same advantageous effects as those of the information processing method are achievable in the following other aspects.

An information processing device according to another aspect of the disclosure includes: a body temperature information acquisition part that acquires body temperature information indicating a body temperature of a user and associated with a user ID specifying the user; a user information acquisition part that acquires user information about the user associated with the user ID; an alert criterion acquisition part that acquires an alert criterion in accordance with the user information from an alert criteria database; a determination part that determines whether the body temperature information satisfies the alert criterion; a generation part that generates alert information for issuing an alert about a health state of the user in association with the user ID when the body temperature information is determined to satisfy the alert criterion; and an output part that outputs the alert information.

A non-transitory computer readable recording medium according to further another aspect of the disclosure stores an information processing program, the information processing program causes a computer to execute: acquiring body temperature information indicating a body temperature of a user and associated with a user ID specifying the user; acquiring user information about the user associated with the user ID; acquiring an alert criterion in accordance with the user information from an alert criteria database; determining whether the body temperature information satisfies the alert criterion; generating alert information for issuing an alert about a health state in association with the user ID when the body temperature information is determined to satisfy the alert criterion; and outputting the alert information.

Embodiments of this disclosure will be described with reference to the accompanying drawings. It should be noted that each of the following embodiments illustrates one example of the disclosure, and does not delimit the technical scope of the disclosure.

### First Embodiment

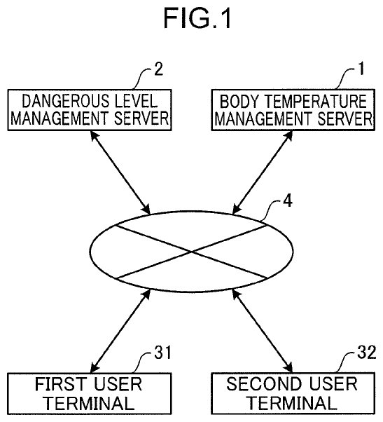

FIG. 1 is a diagram showing an example of a configuration of a body temperature management system in a first embodiment of the disclosure.

The body temperature management system shown in FIG. 1 includes a body temperature management server 1, a dangerous level management server 2, a first user terminal 31, and a second user terminal 32.

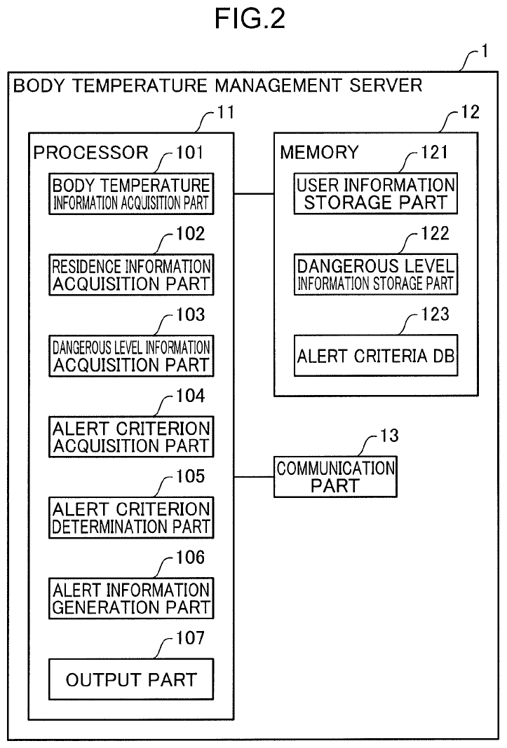

The body temperature management server 1 includes, for example, a cloud server, and is communicably connected to the dangerous level management server 2, the first user terminal 31, and the second user terminal 32 via a network 4. The network 4 includes, for example, the internet. A configuration of the body temperature management server 1 will be described later with reference to FIG. 2

The dangerous level management server 2 generates dangerous level information indicating a dangerous level per region in accordance with a virus spread situation, and transmits the generated dangerous level information to the body temperature management server 1. The dangerous level management server 2 generates dangerous level information indicating a dangerous level, for example, per nation, per prefecture, or per city, town, or village.

The dangerous level is expressed at, for example, six stages of level 5 to level 0. Level 5 indicates the highest dangerous level. The dangerous level is determined, for example, in accordance with the number of persons infected from a specific virus in each region. Examples of the specific virus include a novel coronavirus. For instance, a dangerous level for a region having no new infected person in last four weeks is set to level 0, a dangerous level for a region confirmed to have one or more new infected persons in a single digit on a single day is set to level 1, a dangerous level for a region confirmed to have one or more new infected persons in a single digit for continuous days is set to level 2, a dangerous level for a region confirmed to have new infected persons in a double-digit on a single day is set to level 3, and a dangerous level for a region confirmed to have new infected persons in a double-digit for two or more continuous days is set to level 4, and a dangerous level for a region confirmed to have new infected persons in a triple-digit on a single day is set to level 5. Here, the setting of the dangerous levels is a mere example, and thus is not limited thereto.

The dangerous level management server 2 generates dangerous level information and transmits the generated dangerous level information to the body temperature management server 1, for example, once a day. The dangerous level management server 2 may transmit updated dangerous level information to the body temperature management server 1 at each updating of the dangerous level information.

The first user terminal 31 is, for example, a smartphone, a personal computer, or a tablet-type computer, and is used by a first user. The first user is, for example, an employee of a certain company.

The first user terminal 31 receives an input of user information by the first user, and transmits the received user information to the body temperature management server 1. The user information includes a user ID, a name of the first user, a residence of the first user, an age of the first user, and an underlying disease of the first user. The first user terminal 31 receives an input of the user information at a start of use of the body temperature management system by the first user.

The first user terminal 31 receives an input of a body temperature by the first user, and transmits the received body temperature of the first user to the body temperature management server 1. The first user measures the body temperature thereof with a thermometer and inputs the measured body temperature to the first user terminal 31. When the first user terminal 31 is communicable with the thermometer, the first user terminal 31 may acquire the body temperature of the first user from the thermometer. The first user terminal 31 receives an input of a body temperature of the first user, and transmits the received body temperature of the first user to the body temperature management server 1, for example, once a day.

The second user terminal 32 is, for example, a smartphone, a personal computer, or a tablet-type computer, and is used by a second user. The second user is, for example, an employee of the same company as the first user, and serves as a manager who manages the first user. Specifically, the first user and the second user belong to the same group (department) of the same company, and the second user is a senior and the first user is a junior.

The second user terminal 32 receives an input of user information by the second user, and transmits the received user information to the body temperature management server 1. The second user terminal 32 receives an input of a body temperature by the second user, and transmits the received body temperature of the second user to the body temperature management server 1. The second user terminal 32 further receives body temperature information about the first user from the body temperature management server 1, and displays the received body temperature information about the first user. The second user terminal 32 receives alert information for issuing an alert about a health state of the first user from the body temperature management server 1, and displays the received alert information.

Although the body temperature management system includes the single first user terminal 31 and the single second user terminal 32 in the first embodiment, this disclosure is not particularly limited thereto, and the system may include a plurality of first user terminals 31 and a plurality of second user terminals 32.

FIG. 2 is a diagram showing an example of the body temperature management server 1 in the first embodiment of the disclosure.

The body temperature management server 1 shown in FIG. 2 includes a processor 11, a memory 12, and a communication part 13. The body temperature management server 1 serves as an example of an information processing device.

The processor 11 includes, for example, a CPU (central processing unit). The processor 11 realizes a body temperature acquisition part 101, a residence information acquisition part 102, a dangerous level information acquisition part 103, an alert criterion acquisition part 104, an alert criterion determination part 105, an alert information generation part 106, and an output part 107.

For instance, the memory 12 includes a storage device, such as a RAM (Random Access Memory), an HDD (Hard Disk Drive), an SSD (Solid State Drive), or a flash memory, for storing various kinds of information. The memory 12 realizes a user information storage part 121, a dangerous level information storage part 122, and an alert criteria data base (DB) 123.

The communication part 13 receives user information transmitted from the first user terminal 31 or the second user terminal 32. The communication part 13 stores the received user information in the user information storage part 121. The communication part 13 further receives body temperature information transmitted from the first user terminal 31 or the second user terminal 32. Besides, the communication part 13 receives dangerous level information transmitted from the dangerous level management server 2. The communication part 13 stores the received dangerous level information in the dangerous level information storage part 122.

The user information storage part 121 stores user information per user. Examples of the user information include a user ID specifying a user, a name of the user, a residence of the user, an age of the user, an underlying disease of the user, and a group ID specifying a group to which the user belongs. In the first embodiment, the user information may include at least the residence.

The body temperature acquisition part 101 acquires, from the communication part 13, body temperature information indicating a body temperature of a user and associated with a user ID.

The residence information acquisition part 102 acquires from, the user information storage part 121, residence information indicating a residence of the user and associated with the user ID. The residence information acquisition part 102 serves as an example of a user information acquisition part.

The dangerous level information storage part 122 stores dangerous level information indicating a dangerous level per region in accordance with a virus spread situation.

The dangerous level information acquisition part 103 acquires, from the dangerous level information storage part 122, the dangerous level information indicating the dangerous level per region in accordance with the virus spread situation.

The alert criteria DB 123 stores a plurality of dangerous levels and alert criteria for each body temperature in association with each other.

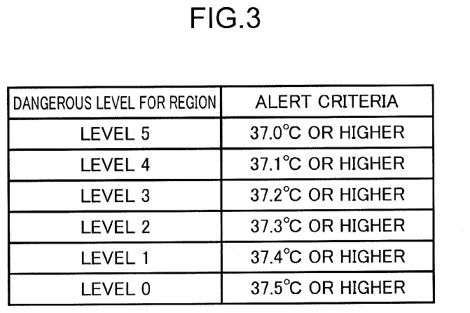

FIG. 3 shows an example of the alert criteria DB 123 in the first embodiment.

As shown in FIG. 3, the alert criteria DB 123 associates a plurality of dangerous levels with alert criteria for each body temperature. For instance, a dangerous level at level 5 is associated with an alert criterion denoting “37° C. or higher”, a dangerous level at level 4 is associated with an alert criterion denoting “37.1° C. or higher”, a dangerous level at level 3 is associated with an alert criterion denoting “37.2° C. or higher”, a dangerous level at level 2 is associated with an alert criterion denoting “37.3° C. or higher”, a dangerous level at level 1 is associated with an alert criterion denoting “37.4° C. or higher”, and a dangerous level at level 0 is associated with an alert criterion denoting “37.5° C. or higher”.

It is seen from this perspective that a body temperature threshold to be an alert criterion is lower as a dangerous level is higher, and the body temperature threshold to be the alert criterion is higher as the dangerous level is lower. This is because a probability of having a fever attributed to a virus infection is higher in a region having a higher dangerous level.

The alert criterion acquisition part 104 acquires an alert criterion in accordance with the user information from the alert criteria DB 123. The alert criterion acquisition part 104 specifies, on the basis of dangerous level information, a specific dangerous level for the residence of the user. The alert criterion acquisition part 104 acquires a specific alert criterion associated with the specified dangerous level from the alert criteria DB 123. For instance, when the dangerous level for the residence of the user is given level 3, the alert criterion acquisition part 104 acquires the alert criterion denoting “37.2° C. or higher”.

The alert criterion determination part 105 determines whether the body temperature information acquired by the body temperature acquisition part 101 satisfies an alert criterion. For example, when the body temperature information indicates 37.6° C. under the alert criterion denoting “37.2° C. or higher”, the alert criterion determination part 105 determines that the body temperature information satisfies the alert criterion. By contrast, for example, when the body temperature information indicates 36.5° C. under the alert criterion denoting “37.2° C. or higher”, the alert criterion determination part 105 determines that the body temperature information dissatisfies the alert criterion.

When the alert criterion determination part 105 determines that the body temperature information satisfies the alert criterion, the alert information generation part 106 generates alert information for issuing an alert about a health state of the user in association with the user ID.

The output part 107 outputs the alert information generated by the alert information generation part 106. The output part 107 outputs the alert information to the communication part 13. The communication part 13 transmits the alert information to the second user terminal 32. When the alert criterion determination part 105 determines that the body temperature information dissatisfies the alert criterion, the output part 107 outputs body temperature information. In this case, the output part 107 outputs the body temperature information to the communication part 13. The communication part 13 transmits the body temperature information to the second user terminal 32.

Next, notification of information by the body temperature management server 1 in the first embodiment of the disclosure will be described.

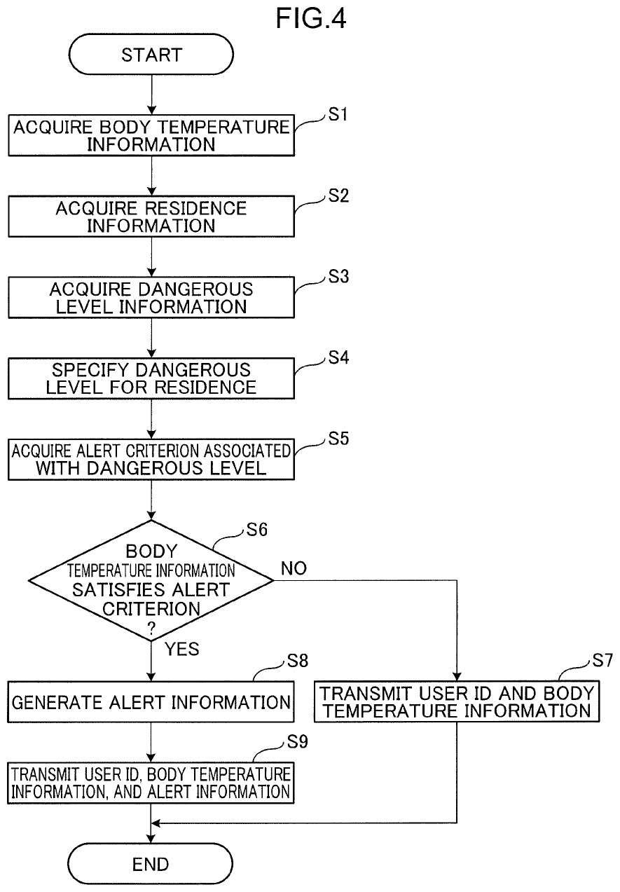

FIG. 4 is a flowchart explaining the notification of information by the body temperature management server 1 in the first embodiment of the disclosure.

First, in step S1, the body temperature acquisition part 101 acquires, from the communication part 13, body temperature information indicating a body temperature of the first user and associated with a user ID. The communication part 13 receives the body temperature information transmitted from the first user terminal 31. The body temperature acquisition part 101 acquires the body temperature information received by the communication part 13.

Next, in step S2, the residence information acquisition part 102 acquires, from the user information storage part 121, residence information indicating a residence of the first user and associated with the user ID. At this time, the residence information acquisition part 102 acquires, from the user information storage part 121, residence information corresponding to the user ID associated with the body temperature information acquired by the body temperature acquisition part 101.

Subsequently, in step S3, the dangerous level information acquisition part 103 acquires, from the dangerous level information storage part 122, dangerous level information indicating a dangerous level per region in accordance with a virus spread situation.

Then, in step S4, the alert criterion acquisition part 104 specifies, on the basis of the dangerous level information, a specific dangerous level for the residence of the first user. The alert criterion acquisition part 104 specifies the dangerous level for the residence of the first user indicated by the residence information acquired by the residence information acquisition part 102 with reference to the dangerous level information acquired by the dangerous level information acquisition part 103.

Next, in step S5, the alert criterion acquisition part 104 acquires a specific alert criterion associated with the specified dangerous level from the alert criteria DB 123.

Subsequently, in step S6, the alert criterion determination part 105 determines whether the body temperature information acquired by the body temperature acquisition part 101 satisfies the alert criterion. At this time, the alert criterion determination part 105 determines whether the body temperature information indicates a value equal to or higher than a body temperature threshold corresponding to a dangerous level. When the body temperature is determined to indicate the value equal to or higher than the body temperature threshold corresponding to the dangerous level, the alert criterion determination part 105 determines that the body temperature information satisfies the alert criterion. By contrast, when the body temperature information is not determined to indicate the value equal to or higher than the body temperature threshold corresponding to the dangerous level, the alert criterion determination part 105 determines that the body temperature information dissatisfies the alert criterion.

Here, when the body temperature information is determined to dissatisfy the alert criterion (NO in step S6), the output part 107 transmits, in step S7, the user ID and the body temperature information to the second user terminal 32 via the communication part 13. The second user terminal 32 is determined to be a transmission destination in advance. The user information includes a group ID specifying a group to which the first user belongs. The memory 12 stores the group ID in association with the second user terminal 32 of the manager (second user) in the group in advance. The output part 107 determines the second user terminal 32 of the second user associated with the group ID of the group to which the first user belongs to be the transmission destination. The second user terminal 32 receives the user ID and the body temperature information. The second user terminal 32 further causes a display part to display the name of the first user corresponding to the user ID, and a body temperature of the first user.

Contrarily, when the body temperature information is determined to satisfy the alert criterion (YES in step S6), the alert information generation part 106 generates, in step S8, alert information for issuing an alert about a health state of the first user in association with the user ID.

Next, in step S9, the output part 107 transmits, to the second user terminal 32, the user ID, the body temperature information, and the alert information generated by the alert information generation part 106 via the communication part 13. The second user terminal 32 is determined to be the transmission destination in advance as described above. The second user terminal 32 receives the user ID, the body temperature information, and the alert information. The second user terminal 32 further causes the display part to display the name of the first user corresponding to the user ID, and a body temperature of the first user, and the alert information.

In this way, alert information for issuing an alert about a health state of a user is output when an alert criterion is acquired in accordance with user information from the alert criteria DB 123 and body temperature information is determined to satisfy the alert criterion. This achieves presentation of alert information in accordance with an environment around the user or a situation of the user.

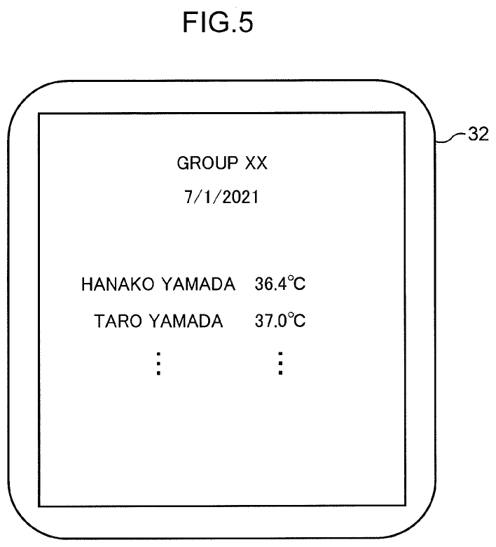

FIG. 5 shows an example of a body temperature presentation screen image displayed on the second user terminal 32 in the first embodiment.

As shown in FIG. 5, when the body temperature information dissatisfies an alert criterion, the second user terminal 32 displays a body temperature presentation screen image including: a name of a group to which the second user belongs; a date; a name of a first user being another member belonging to the group; and a body temperature of the first user. The second user terminal 32 stores the name of the group to which the second user belongs, and a name and a user ID of a member belonging to the group in advance. The second user terminal 32 generates and displays the body temperature presentation screen image when receiving the user ID and the body temperature information from the body temperature management server 1.

It is seen from FIG. 5 that the group has the name “group XX”, the date is “Jul. 1, 2021”, the group includes first users named “Hanako YAMADA” and “Taro YAMADA”, and the first users respectively have body temperatures indicating “36.4° C.” and “37.0° C.”.

The second user can check each body temperature of each first user belonging to the same group by seeing the body temperature presentation screen image displayed on the second user terminal 32.

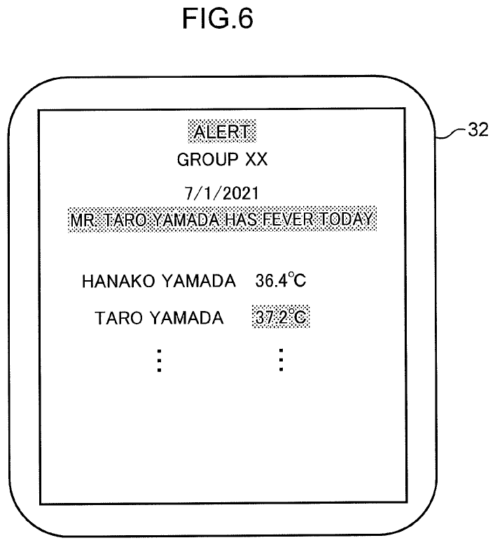

FIG. 6 shows an example of an alert presentation screen image displayed on the second user terminal 32 in the first embodiment.

As shown in FIG. 6, when the body temperature information satisfies the alert criterion, the second user terminal 32 displays an alert presentation screen image including: a name of a group to which the second user belongs; a date; a name of a first user being another member belonging to the group; a body temperature of the first user; and alert information. The second user terminal 32 stores the name of the group to which the second user belongs, and a name and a user ID of a member belonging to the group in advance. The second user terminal 32 generates and displays the alert presentation screen image when receiving the user ID, the body temperature information, and the alert information from the body temperature management server 1.

It is seen from FIG. 6 that the group has the name “group XX”, the date is “Jul. 1, 2021”, the group includes first users named “Hanako YAMADA” and “Taro YAMADA”, and the first users respectively have body temperatures indicating “36.4° C.” and “37.2° C.”, and the alert information includes a sentence saying, “Mr. Taro YAMADA has a fever today.” The second user terminal 32 displays alert information for issuing an alert indicating that the first user has a fever. It is not that the alert information is always displayed when a body temperature of a first user exceeds 37.0° C., but the alert information is displayed when the body temperature of the first user satisfies the alert criterion corresponding to the dangerous level for the residence.

The second user can check the body temperature of the first user belonging to the same group and confirm the necessity of attention to the health condition of the first user by seeing the alert presentation screen image displayed on the second user terminal 32.

Although the alert criteria DB 123 associates a dangerous level with an alert criterion in the first embodiment, this disclosure is not particularly limited thereto, and the alert criteria DB may associate an alert criterion with a combination of a dangerous level with at least one of presence or absence of an underlying disease, a type of the underlying disease, and an age.

### Second Embodiment

In the first embodiment, the alert criteria data base associates a plurality of dangerous levels with alert criteria for each body temperature, a specific dangerous level for a residence of a user is specified on the basis of dangerous level information, and a specific alert criterion associated with the specified dangerous level is acquired from the alert criteria data base. By contrast, in a second embodiment, an alert criteria data base associates the number of contact persons with an alert criterion for a body temperature, and the alert criterion is acquired from the alert criteria database in association with the number of contact persons with a user on a previous day.

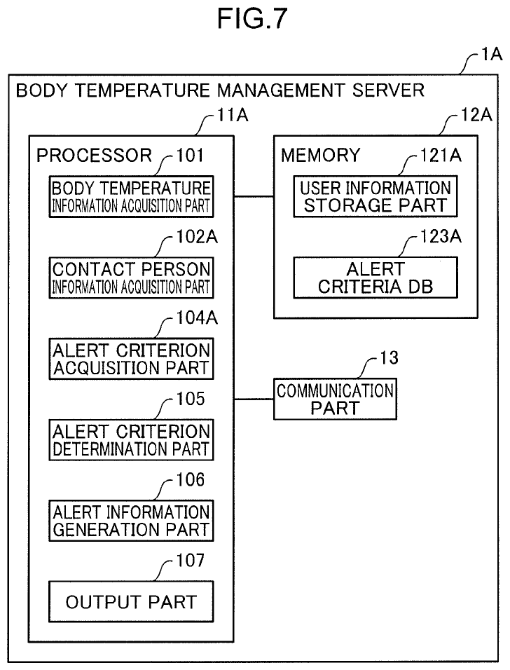

FIG. 7 is a diagram showing an example of a body temperature management server 1A in the second embodiment of the disclosure. A body temperature management system in the second embodiment includes the body temperature management server 1A, a first user terminal 31, and the second user terminal 32, while excluding the dangerous level management server 2.

The body temperature management server 1A shown in FIG. 7 includes a processor 11A, a memory 12A, and a communication part 13. The body temperature management server 1A serves as an example of the information processing device.

The processor 11A realizes a body temperature acquisition part 101, a contact person information acquisition part 102A, an alert criterion determination part 104A, an alert criterion determination part 105, an alert information generation part 106, and an output part 107. The memory 12A realizes a user information storage part 121A and an alert criteria data base (DB) 123A. In the second embodiment, elements which are the same as those in the first embodiment are given the same reference signs and numerals, and thus explanation therefor will be omitted.

The user information storage part 121A stores user information per user. Examples of the user information include a user ID specifying a user, a name of the user, a residence of the user, an age of the user, an underlying disease of the user, a group ID specifying a group to which the user belongs, and the number of contact persons with the user on a previous day. In the second embodiment, the user information may include at least the number of contact persons on a previous day.

The first user terminal 31 counts the number of contact persons with a first user. For instance, the first user terminal 31 increases the number of contact persons through increment when another user terminal stays in a range of one meter from the first user terminal for fifteen minutes or longer. The first user terminal 31 transmits the number of contact persons per day to the body temperature management server 1A. For instance, the first user terminal 31 may transmit the number of contact persons on a previous day to the body temperature management server 1A when the next day comes. The communication part 13 receives the number of contact persons on the previous day transmitted from the first user terminal 31, and stores the received number of contact persons on the previous day in the user information storage part 121A.

When a contact person management server which manages the number of contact persons with a user is communicably connected to the body temperature management server 1A, the communication part 13 may receive the number of contact persons with the first user on a previous day from the contact person management server.

The contact person information acquisition part 102A acquires, from the user information storage part 121A, contact person information indicating the number of contact persons with a user associated with a user ID. The contact person information acquisition part 102A serves as an example of the user information acquisition part.

The alert criteria DB 123A stores the number of contact persons and an alert criterion for a body temperature in association with each other.

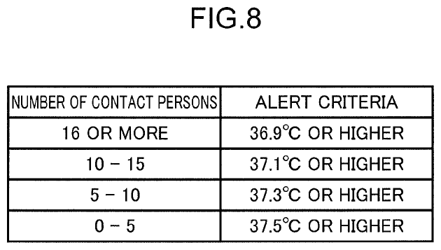

FIG. 8 shows an example of the alert criteria DB 123A in the second embodiment.

As shown in FIG. 8, the alert criteria DB 123A associates the number of contact persons with an alert criterion for a body temperature. For example, the number of sixteen or more contact persons is associated with an alert criterion denoting “36.9° C. or higher”, the number of ten to fifteen contact persons is associated with an alert criterion denoting “37.1° C. or higher”, the number of five to ten contact persons is associated with an alert criterion denoting “37.3° C. or higher”, and the number of zero to five contact persons is associated with an alert criterion denoting “37.5° C. or higher”.

It is seen from this perspective that a body temperature threshold to be an alert criterion is lower as the number of contact persons is larger, and the body temperature threshold to be the alert criterion is higher as the number of contact persons is smaller. This is because a probability of having a fever attributed to a virus infection is higher as the number of contact persons is larger.

The alert criterion acquisition part 104A acquires, from the alert criteria DB 123A, an alert criterion in association with the number of contact persons with the user on a previous day acquired by the contact person information acquisition part 102A. For instance, when the number of contact persons with the user on the previous day indicates “12”, the alert criterion acquisition part 104A acquires the alert criterion denoting “37.1° C. or higher”.

Next, notification of information by the body temperature management server 1A in the second embodiment of the disclosure.

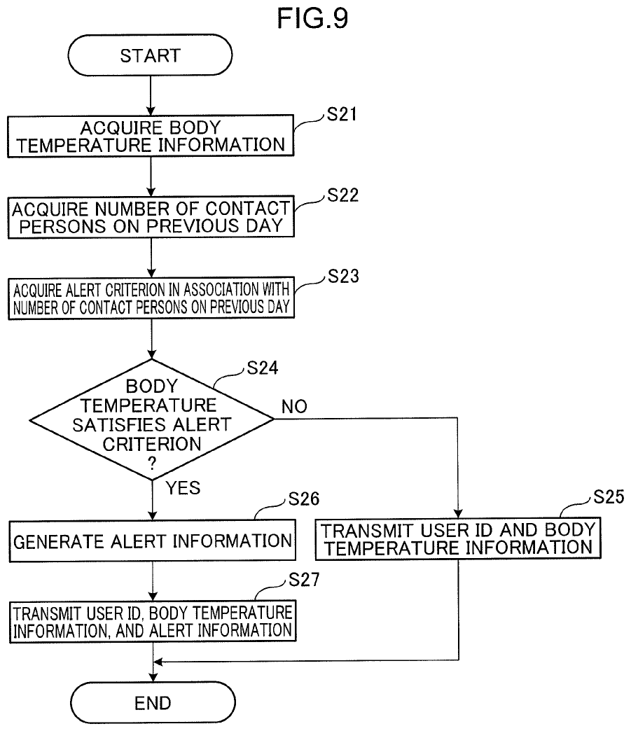

FIG. 9 is a flowchart explaining the notification of information by the body temperature management server 1A in the second embodiment of the disclosure.

First, in step S21, the body temperature acquisition part 101 acquires, from the communication part 13, body temperature information indicating a body temperature of the first user and associated with a user ID. The communication part 13 receives the body temperature information transmitted from the first user terminal 31. The body temperature acquisition part 101 acquires the body temperature information received by the communication part 13.

Next, in step S22, the contact person information acquisition part 102A acquires, from the user information storage part 121A, contact person information indicating the number of contact persons with the first user on a previous day and associated with the user ID. At this time, the contact person information acquisition part 102A acquires, from the user information storage part 121A, the number of contact persons on a previous day corresponding to the user ID associated with the body temperature information acquired by the body temperature acquisition part 101. The contact person information indicating the number of contact persons with the first user on the previous day may be transmitted to the first user terminal 31 together with the body temperature information. In this case, the communication part 13 may receive the contact person information together with the body temperature information. The contact person information acquisition part 102A may acquire, from the communication part 13, the contact person information indicating the number of contact persons with the first user on the previous day.

Subsequently, in step S23, the alert criterion acquisition part 104A acquires, from the alert criteria DB 123A, an alert criterion in association with the number of contact persons on the previous day acquired by the contact person information acquisition part 102A.

Here, steps S24 to S27 are the same as steps S6 to S9 in FIG. 4, and thus the descriptions therefor are omitted.

Although the alert criteria DB 123A associates the number of contact persons on a previous day with an alert criterion in the second embodiment, this disclosure is not particularly limited thereto, and the alert criteria DB may associate an alert criterion with a combination of the number of contact persons on the previous day with at least one of a dangerous level for a residence, presence or absence of an underlying disease, a type of the underlying disease, and an age.

### Third Embodiment

In the first embodiment, the alert criteria data base associates a plurality of dangerous levels with alert criteria for each body temperature, a specific dangerous level for a residence of a user is specified on the basis of dangerous level information, and a specific alert criterion associated with the specified dangerous level is acquired from the alert criteria data base. By contrast, in a third embodiment, an alert criteria database includes a first alert criterion for a body temperature, and a second alert criterion for at least one of: a health condition; a fever continuous day number indicating the number of days when the body temperature continuously shows a predetermined temperature or higher; a dangerous level per region in accordance with a virus spread situation; an underlying disease; an age; and the number of contact persons on a previous day. Furthermore, body temperature information is determined whether to satisfy the first alert criterion, and at least one of health condition information, the fever continuous day number, the dangerous level in the residence, underlying disease information, age information, and contact person information is determined whether to satisfy the second alert criterion. Alert information is generated when the body temperature information is determined to satisfy the first alert criterion, and the at least one of the health condition information, the fever continuous day number, the dangerous level in the residence, the underlying disease information, the age information, and the contact person information is determined to satisfy the second alert criterion.

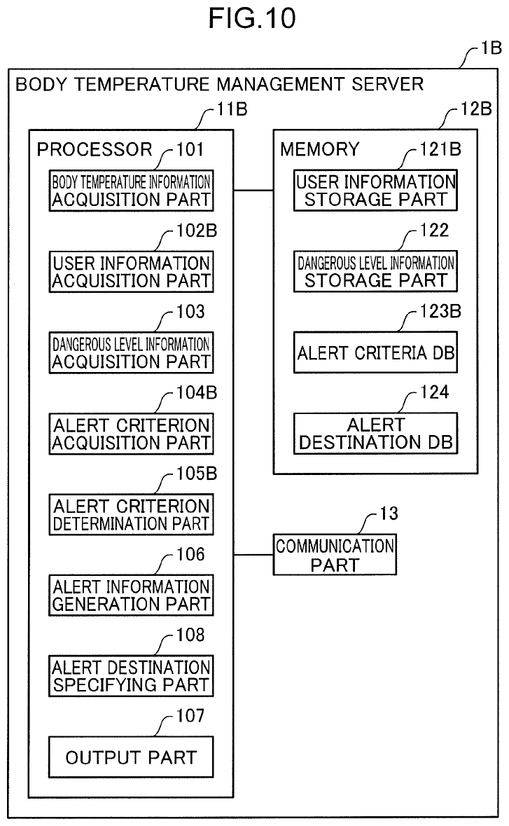

FIG. 10 is a diagram showing an example of a body temperature management server 1B in the third embodiment of the disclosure.

The body temperature management server 1B shown in FIG. 10 includes a processor 11B, a memory 12B and a communication part 13. The body temperature management server 1B serves as an example of the information processing device.

The processor 11B realizes a body temperature acquisition part 101, a user information acquisition part 102B, a dangerous level information acquisition part 103, an alert criterion determination part 104B, an alert criterion determination part 105B, an alert information generation part 106, an output part 107, and an alert destination specifying part 108. The memory 12B realizes a user information storage part 121B, a dangerous level information storage part 122, an alert criteria data base (DB) 123B, and an alert destination database (DB) 124. In the third embodiment, elements which are the same as those in the first embodiment are given the same reference signs and numerals, and thus explanation therefor will be omitted.

The user information storage part 121B stores user information per user. Examples of the user information include a user ID specifying a user, a name of the user, a past body temperature history of the user, health condition information indicating a health condition of the user, a residence of the user, attendance information about an attendance situation of the user, an age of the user, an underlying disease of the user, a group ID specifying a group to which the user belongs, and the number of contact persons with the user on a previous day.

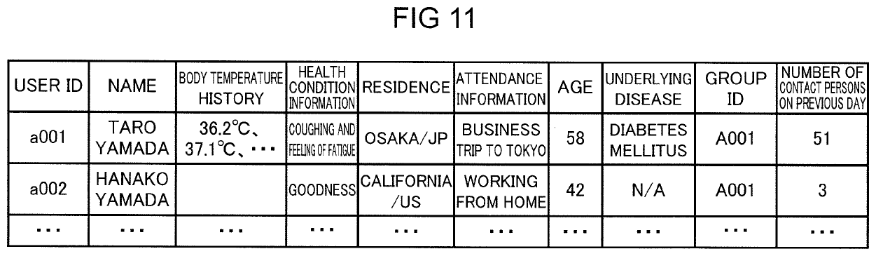

FIG. 11 shows an example of the user information in the third embodiment.

A body temperature history includes, for example, body temperature values of the user for one week.

The health condition information indicates a health condition of the user, such as, coughing, feeling of fatigue, or goodness. The communication part 13 receives health condition information input by the user from a first user terminal 31 or a second user terminal 32. The first user terminal 31 receives an input of health condition information by a first user or the second user terminal 32 receives an input of health condition information by a second user. The first user terminal 31 or the second user terminal 32 receives a selection of at least one item by the first user or the second user from among a plurality of items each representing a health condition. The items include, for example, goodness, coughing, feeling of fatigue, a sore throat, diarrhea, a headache, and a taste disorder. The communication part 13 may receive the health condition information together with the body temperature information, or may receive the health condition information independently of the body temperature information.

The attendance information represents an attendance schedule of the user, such as, attending, working from home, or a business trip. The communication part 13 receives the attendance information input by the user from the first user terminal 31 or the second user terminal 32. The communication part 13 may receive schedule information stored in the first user terminal 31 or the second user terminal 32, and may store, in the user information storage part 121B, the attendance information included in the received schedule information.

Although the user information includes the health condition information, the body temperature history, the residence information, the underlying disease information, the age information, and the contact person information in the third embodiment, this disclosure is not limited thereto, and the user information may include at least one of the health condition information, the body temperature history, the residence information, the underlying disease information, the age information, and the contact person information. The user information may further include other information in addition to the aforementioned information.

The user information acquisition part 102B acquires, from the user information storage part 121B, user information associated with a user ID. The user information acquisition part 102B acquires, from the user information acquisition part 121B, body temperature history information, health condition information, residence information, age information, underlying disease information, and contact person information.

The alert criteria DB 123B includes the first alert criterion for the body temperature, and the second alert criterion for: a health condition; a fever continuous day number indicating the number of days when the body temperature continuously shows a predetermined temperature or higher; a dangerous level per region in accordance with a virus spread situation; an underlying disease; an age; and the number of contact persons on ae previous day.

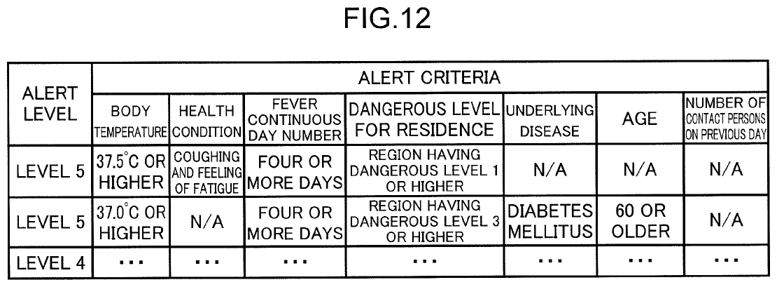

FIG. 12 shows an example of the alert criteria DB 123B in the third embodiment.

As shown in FIG. 12, the alert criteria DB 123B associates an alert level with an alert criterion. The alert level is expressed at, for example, six stages of level 5 to level 0. The alert level is higher as a possibility of infection from a specific virus is higher. Besides, the alert level is available for determination of a notification destination of alert information, and thus a user at a notification destination is changeable in accordance with the alert level.

Alert criteria include a first alert criterion for the body temperature, and a second alert criterion for: a health condition; a fever continuous day number indicating the number of days when the body temperature continuously shows a predetermined temperature or higher; a dangerous level per region in accordance with a virus spread situation; an underlying disease; an age; and the number of contact persons on a previous day.

For instance, the alert criteria DB 123B includes a first alert criterion for a body temperature denoting 37.5° C. or higher, and a second alert criterion for: health condition information indicating coughing and feeling of fatigue: a fever continuous day number of four or more days; and a residence in a region having a dangerous level 1 or higher. Alternatively, for instance, the alert criteria DB 123B includes a first alert criterion for a body temperature denoting 37.0° C. or higher, and a second alert criterion for: a fever continuous day number of four or more days; a residence in a region having a dangerous level 3 or higher; an underlying disease of diabetes mellitus; and an age of sixty or older.

In the third embodiment, the alert criteria DB 123B includes the first alert criterion for the body temperature, and the second alert criterion for at least one of: the health condition; the fever continuous day number; the dangerous level; the underlying disease; the age; and the number of contact persons on the previous day.

The alert criterion acquisition part 104B acquires alert criteria including the first alert criterion and the second alert criterion from the alert criteria DB 123B. The alert criterion acquisition part 104B acquires a specific one alert criterion among the alert criteria. At this time, the alert criterion acquisition part 104B firstly acquires an alert criterion associated with the highest alert level. Thereafter, the alert criterion acquisition part 104B acquires an alert criterion in descending order of the alert level per determination of dissatisfying the alert criterion.

The alert criterion determination part 105B determines whether the body temperature information satisfies the first alert criterion, and determines whether at least one of the health condition information, the fever continuous day number, the dangerous level in the residence, the underlying disease information, the age information, and the contact person information satisfies the second alert criterion.

The alert information generation part 106 generates alert information when the alert criterion determination part 105B determines that the body temperature information satisfies the first alert criterion, and that the at least one of the health condition information, the fever continuous day number, the dangerous level in the residence, the underlying disease information, the age information, and the contact person information satisfies the second alert criterion.

The alert destination DB 124 stores an alert level and an alert destination in association with each other.

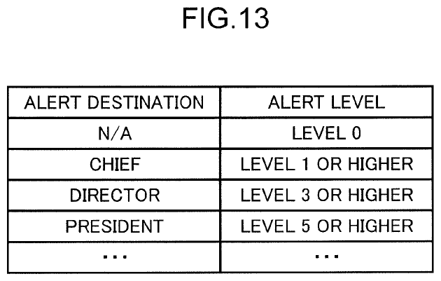

FIG. 13 shows an example of the alert destination DB 124 in the third embodiment.

As shown in FIG. 13, an alert level at level 5 or higher is associated with a president as a user at an alert destination, an alert level at level 3 or higher is associated with a director as a user at an alert destination, an alert level at level 1 or higher is associated with a chief as a user at an alert destination, and an alert level at level 0 is associated with no user at an alert destination.

The alert destination specifying part 108 specifies an alert level associated with the alert criterion when the body temperature information is determined to satisfy the alert criterion. The alert destination specifying part 108 further specifies, with reference to the alert destination DB 124 associating the alert level with an alert destination, the alert destination associated with the specified alert level.

The alert destination specifying part 108 specifies the president as the user at the alert destination when the alert level indicates level 5 or higher, specifies the director as the user at the destination when the alert level indicates level 3 or higher, specifies the chief as the user at the alert destination when the alert level indicates the level 1 or higher, and specifies no one as the user at the alert destination when the alert level indicates level 0.

The output part 107 outputs the alert information to the alert destination specified by the alert destination specifying part 108. For instance, the output part 107 transmits the alert information to the second user terminal 32 of the president when the alert destination specifying part 108 specifies the president as the alert destination.

The president represents a manager of a first group to which all the employees in a company belongs, the director represents a manager of a second group of employees belonging to a specific department in the company, and a chief is a manger of a third group of employees belonging to a specific division in the department. It is seen from this perspective that the number of employees belonging to the first group is larger than the number of employees belonging to the second group. Moreover, the number of employees belonging to the second group is larger than the number of employees belonging to the third group. Transmission of alert information about users each having an alert level at level 1 or higher to the president would result in a huge amount of the alert information. This makes the work of confirming the information by the president complicated. Hence, only alert information about each user having an alert level at level 5 or higher is transmitted to the president who has to manage the large number of users. In this manner, notification of only the alert information about each user having a high possibility of virus infection is achieved.

Next, notification of information by the body temperature management server 1B in the third embodiment of the disclosure will be described.

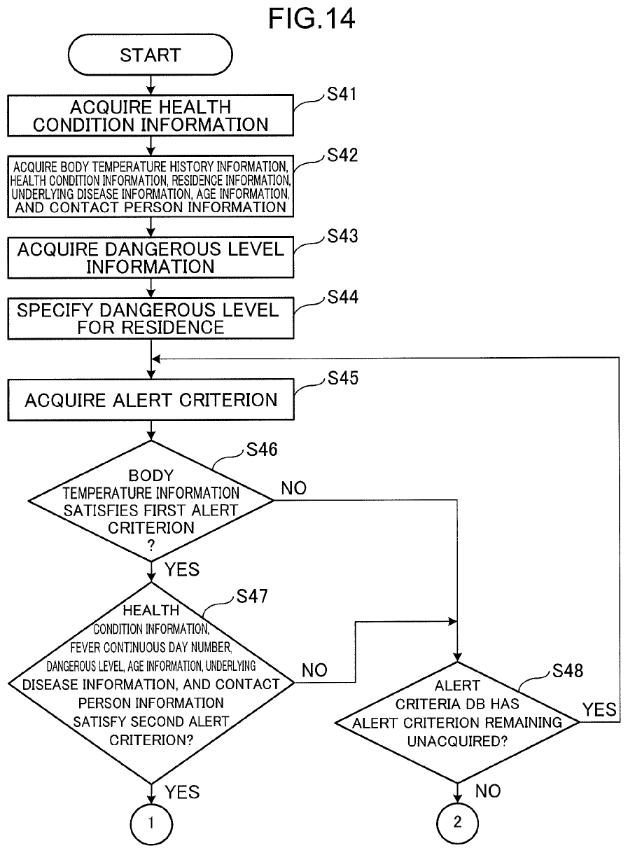

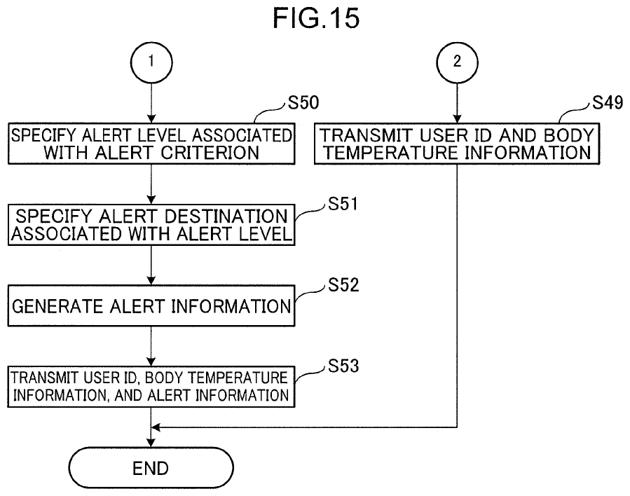

FIG. 14 is a first flowchart explaining the notification of information by the body temperature management server 1B in the third embodiment of the disclosure. FIG. 15 is a second flowchart explaining the notification of information by the body temperature management server 1B in the third embodiment of the disclosure.

First, in step S41, the body temperature acquisition part 101 acquires, from the communication part 13, body temperature information indicating a body temperature of the first user and associated with a user ID. The communication part 13 receives the body temperature information transmitted from the first user terminal 31. The body temperature acquisition part 101 acquires the body temperature information received by the communication part 13.

Next, in step S42, the user information acquisition part 102B acquires, from the user information acquisition part 121B, body temperature history information, health condition information, residence information, age information, underlying disease information, and contact person information respectively associated with the user ID.

Subsequently, in step S43, the dangerous level information acquisition part 103 acquires, from the dangerous level information storage part 122, dangerous level information indicating a dangerous level per region in accordance with a virus spread situation.

Then, in step S44, the alert criterion acquisition part 104B specifies, on the basis of the dangerous level information, a specific dangerous level for the residence of the first user. The alert criterion acquisition part 104B specifics the dangerous level for the residence of the first user indicated by the residence information acquired by the user information acquisition part 102B with reference to the dangerous level information acquired by the dangerous level information acquisition part 103.

Next, in step S45, the alert criterion acquisition part 104B acquires, from the alert criteria DB 123B, a specific one alert criterion among a plurality of alert criteria. The alert criteria include the first alert criterion for use in determination on body temperature information, and the second alert criterion for use in determination on health condition information, the fever continuous day number, a dangerous level, age information, underlying disease information, and contact person information.

Subsequently, in step S46, the alert criterion determination part 105B determines whether the body temperature information acquired by the body temperature acquisition part 101 satisfies the first alert criterion. Here, the alert criterion determination part 105B determines whether the body temperature information indicates a body temperature threshold or higher. When the body temperature is determined to indicate the body temperature threshold or higher, the alert criterion determination part 105B determines that the body temperature information satisfies the first alert criterion. Contrarily, when the body temperature is not determined to indicate the body temperature threshold or higher, the alert criterion determination part 105B determines that the body temperature information dissatisfies the first alert criterion.

Here, when the body temperature information is determined to dissatisfy the first alert criterion (NO in step S46), the flow proceeds to step S48.

Contrarily, when the body temperature information is determined to satisfy the first alert criterion (YES in step S46), the alert criterion determination part 105B determines, in step S47, whether the health condition information, the fever continuous day number, the dangerous level, the age information, the underlying disease information, and the contact person information satisfy the second alert criterion. The alert criterion determination part 105B calculates, on the basis of the body temperature history information, the fever continuous day number showing a body temperature continuously indicating a predetermined temperature or higher. The predetermined temperature indicates, for example, 37.0° C. defined as a determination criterion for having a fever. The predetermined temperature may indicate the same value as the body temperature threshold of the first alert criterion.

At this time, the alert criterion determination part 105B determines whether the health condition information indicates a predetermined health condition, determines whether the fever continuous day number indicates a predetermined number of days or more, determines whether the dangerous level indicates a predetermined level or higher, determines whether the age information indicates a predetermined age or older, determines whether the underlying disease information indicates a predetermined underlying disease, and determines whether the number of contact persons on a previous day indicated by the contact person information indicates a predetermined number. When results of determination on the health condition information, the fever continuous day number, the dangerous level, the age information, the underlying disease information, and the contact person information are all affirmative, the alert criterion determination part 105B determines that the health condition information, the fever continuous day number, the dangerous level, the age information, the underlying disease information, and the contact person information satisfy the second alert criterion. By contrast, when at least one of the results of determination on the health condition information, the fever continuous day number, the dangerous level, the age information, the underlying disease information, and the contact person information is negative, the alert criterion determination part 105B determines that the health condition information, the fever continuous day number, the dangerous level, the age information, the underlying disease information, and the contact person information dissatisfy the second alert criterion.

Here, the second alert criterion does not always fully cover determination criteria for the health condition information, the fever continuous day number, the dangerous level, the age information, the underlying disease information, and the contact person information. For instance, the second alert criterion shown in the first record in FIG. 12 covers determination criteria for the health condition information, the fever continuous day number, and the dangerous level, but fails to cover determination criteria for the age information, the underlying disease information, and the contact person information. Specifically, the second alert criterion may cover at least one of the determination criteria for the health condition information, the fever continuous day number, the dangerous level, the age information, the underlying disease information, and the contact person information.

When it is determined that the health condition information, the fever continuous day number, the dangerous level, the age information, the underlying disease information, and the contact person information dissatisfy the second alert criterion (NO in step S47), the alert criterion acquisition part 104B determines, in step S48, whether the alert criteria DB 123B has any alert criterion remaining unacquired.

When it is determined that the alert criteria DB 123B has some alert criterion remaining unacquired (YES in step S48), the flow returns to step S45, and the alert criterion acquisition part 104B further acquires, from the alert criteria DB 123B, one alert criterion remaining unacquired among the plurality of alert criteria.

Contrarily, when the alert criteria DB 123B is determined to have no alert criterion remaining unacquired (NO in step S48), the output part 107 transmits, in step S49, the user ID and the body temperature information to the second user terminal 32 via the communication part 13. A user terminal to be a transmission destination is determined in advance. The user information includes a group ID specifying a group to which the first user belongs. The memory 12 stores the group ID in association with the user terminal of the manager (second user) in the group in advance. The output part 107 determines the second user terminal 32 of the second user associated with the group ID of the group to which the first user belongs to be the transmission destination. The second user terminal 32 receives the user ID and the body temperature information. The second user terminal 32 further causes a display part to display the name of the first user corresponding to the user ID, and a body temperature of the first user.

The first user may belong to a plurality of groups. The output part 107 may determine a plurality of second user terminals 32 of a plurality of second users respectively associated with group IDs of the groups to which the first user belongs to be transmission destinations. The second users include, for example, the president of the company where the first user works, a director of a specific department in the company, and a chief of a specific division in the department.

By contrast, when it is determined that the health condition information, the fever continuous day number, the dangerous level, the age information, the underlying disease information, and the contact person information satisfy the second alert criterion (YES in step S47), the alert destination specifying part 108 specifies, in step S50, a specific alert level associated with the acquired alert criterion with reference to the alert criteria DB 123B.

Next, in step S51, the alert destination specifying part 108 specifies, with reference to the alert destination DB 124, an alert destination associated with the specified alert level.

Subsequently, in step S52, the alert information generation part 106 generates alert information for issuing an alert about a health state of the first user in association with the user ID.

Then, in step 53, the output part 107 transmits the user ID, the body temperature information, and the alert information generated by the alert information generation part 106 to the second user terminal 32 at the alert destination specified by the alert destination specifying part 108 via the communication part 13. The second user terminal 32 receives the user ID, the body temperature information, and the alert information. The second user terminal 32 further causes the display part to display the name of the first user corresponding to the user ID, and a body temperature of the first user, and the alert information.

The alert destination is not limited to the single second user terminal 32. A plurality of second user terminals 32 may serve as alert destinations.

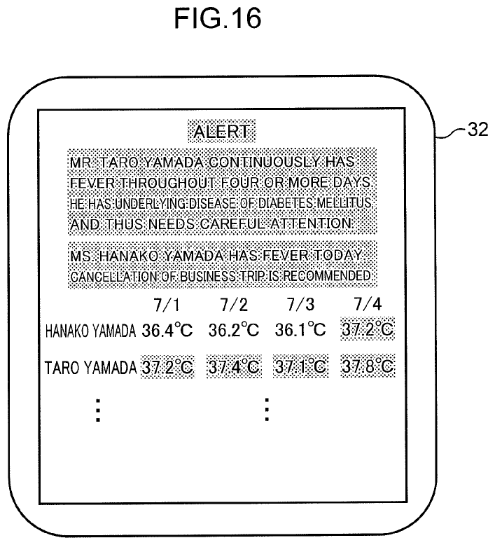

FIG. 16 shows an example of an alert presentation screen image displayed on the second user terminal 32 in the third embodiment.

As shown in FIG. 16, when the body temperature information satisfies the first alert criterion, and the health condition information, the fever continuous day number, the dangerous level, the age information, the underlying disease information, and the contact person information satisfy the second alert criterion, the second user terminal 32 displays an alert presentation screen image including: the name of the first user; body temperature values of the first user in a period of four days; and alert information. The second user terminal 32 stores the name of the group to which the second user belongs, and a name and a user ID of a member belonging to the group in advance. The second user terminal 32 generates and displays the alert presentation screen image when receiving the user ID, the body temperature information, and the alert information from the body temperature management server 1B.

It is seen from FIG. 16 that the second user is a director, the group managed by the second user includes first users named “Hanako YAMADA” and “Taro YAMADA”, and an alert level associated with an alert criterion to be satisfied by the body temperature and the user information about “Hanako YAMADA” is given level 3, and an alert level associated with an alert criterion to be satisfied by the body temperature and the user information about “Taro YAMADA” is given level 5. The alert presentation screen image may show body temperature values in a period of four days from July 1^{st} to July 4^{th}. The alert presentation screen image may show the name of the group.

Since the second user is the director, the alert information about “Hanako YAMADA” and “Taro YAMADA” each being the first user having the alert level at level 3 or higher is displayed on the alert presentation screen image. The alert information about “Taro YAMADA” includes a set of sentences saying, “Mr. Taro YAMADA continuously has a fever throughout four or more days. He has an underlying disease of diabetes mellitus, and thus needs careful attention.” Moreover, the alert information about “Hanako YAMADA” includes a set of sentences saying, “Ms. Hanako YAMADA has a fever today. Cancelation of the business trip is recommended.”

The second user terminal 32 displays the alert information for issuing an alert for “Taro YAMADA” having the fever throughout the four or more days, and issuing an alert for the underlying disease of diabetes mellitus of “Taro YAMADA”. The second user terminal 32 further issues an alert for the fever of “Hanako YAMADA” and displays alert information for recommending cancelation of the business trip.

The user information includes attendance information representing an attendance schedule of the first user. Hence, the alert information generation part 106 may recommend cancellation of a business trip to the first user or encourage the first user to work from home when generating alert information. The alert information generation part 106 may calculate a severity rate from the type of the underlying disease and the age of the first user, and encourage the first user to work from home when the calculated severity rate indicates a predetermined value or higher.

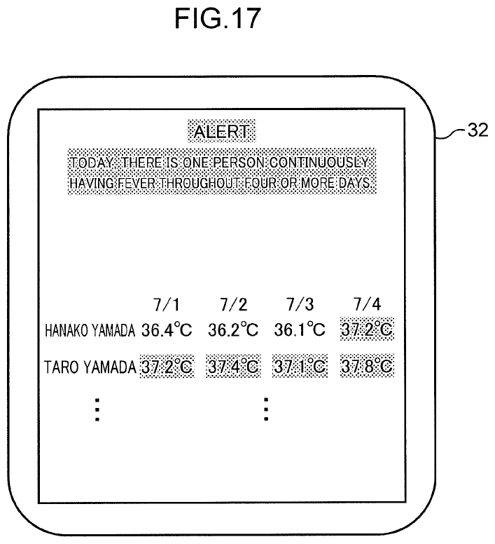

FIG. 17 shows another example of the alert presentation screen image displayed on the second user terminal 32 in the third embodiment of the disclosure.

As shown in FIG. 17, when the body temperature information satisfies the first alert criterion, and the health condition information, the fever continuous day number, the dangerous level, the age information, the underlying disease information, and the contact person information satisfy the second alert criterion, the second user terminal 32 displays an alert presentation screen image including the name of the first user, body temperature values of the first user in a period of four days, and alert information. The second user terminal 32 stores the name of the group to which the second user belongs, and a name and a user ID of a member belonging to the group in advance. The second user terminal 32 generates and displays the alert presentation screen image when receiving the user ID, the body temperature information, and the alert information from the body temperature management server 1B.

It is seen from FIG. 17 that the second user is the president, the group managed by the second user includes first users named “Hanako YAMADA” and “Taro YAMADA”, and an alert level associated with an alert criterion to be satisfied by the body temperature and the user information about “Hanako YAMADA” is given level 3, and an alert level associated with an alert criterion to be satisfied by the body temperature and the user information about “Taro YAMADA” is given level 5. The alert presentation screen image may show body temperature values in a period of four days from July 1^{st} to July 4^{th}. The alert presentation screen image may show the name of the group.

Since the second user is the president, the alert information about “Taro YAMADA” being the first user having the alert level at level 5 or higher is displayed on the alert presentation screen image. The alert information about “Taro YAMADA” includes a sentence saying, “Today, there is one person continuously having a fever throughout four or more days.”

The second user terminal 32 displays alert information for issuing an alert for one person continuously having a fever throughout four or more days. Here, the alert information generation part 106 may change the sentence of the alert information in accordance with an attribute (position) of the second user at the alert destination or with the number of first users managed by the second user.

The alert information generation part 106 may generate alert information in a display manner in accordance with a specified alert level. For instance, the alert information generation part 106 may generate alert information having a distinctive color in accordance with an alert level. For instance, the alert information generation part 106 may display characters in a sentence of alert information for alert levels 1 and 2 in a blue color, and may display characters in a sentence of alert information for alert levels 3 and 4 in a yellow color, and may display characters in a sentence of alert information for alert level 5 in a red color. Alternatively, for example, the alert information generation part 106 may display a background of the alert information for alert levels 1 and 2 in a blue color, display a background of the alert information for alert levels 3 and 4 in a yellow color, and display a background of alert information for alert level 5 in a red color.

The alert criteria DB 123, 123A may further store an alert criterion and an alert level in association with each other in the first and second embodiments as well. The processor 11, 11A of the body temperature management server 1, 1A may further include an alert destination specifying part 108, and the memory 12, 12A may further include an alert destination DB 124. In this case, when body temperature information is determined to satisfy an alert criterion, the alert destination specifying part 108 may specify an alert level associated with the alert criterion, and specifies, with reference to the alert destination DB 124, an alert destination associated with the specified alert level. The output part 107 may output alert information to the alert destination specified by the alert destination specifying part 108.

The first user terminal may receive an input of vaccination information against a virus infection by the first user, and transmit the vaccination information against the virus infection to the body temperature management server. At this time, the body temperature management server may set the vaccination information to an alert criterion and change an alert level in accordance with the vaccination information.

The body temperature management server may further issue an alert when there is a given number of persons or more each having a body temperature exceeding a threshold within a range of a specific place. For instance, the body temperature management server may issue an alert to an event holder when there is a given number of persons or more each having a body temperature exceeding a threshold in an event hall like a concert hall.

When the body temperature management server 1 does not receive a measured body temperature from the first user terminal 31 of the first user and receives a measured body temperature from another user terminal of another member in a group to which the first user belongs, the measurement of the body temperature by another member may be notified to the first user terminal 31. When the body temperature management server 1 does not receive a measured body temperature from the first user terminal 31 of the first user and a body temperature of another member in a group to which the first user belongs is continuously measured throughout a predetermined period, the continuous measurement of the body temperature by another member throughout the predetermined period may be notified to the first user terminal 31.

The body temperature management server 1 may calculate a temperature measurement rate of the group to which the first user belongs on an exact day, and transmit the calculated temperature measurement rate to the first user terminal 31.

When the body temperature management server 1 does not receive a measured body temperature from the first user terminal 31 of the first user and receives a measured temperature from a user terminal of another user whose action pattern of temperature measurement is similar to an action pattern of temperature measurement of the first user, the measurement of the body temperature by another user whose action pattern of temperature measurement is similar to the action pattern of temperature measurement of the first user may be notified to the first user terminal 31. Such another user whose action pattern of temperature measurement is similar to the action pattern of temperature measurement of the first user represents, for example, another user whose temperature measurement rate in a predetermined period is similar to a temperature measurement rate of the first user in the period, or another user whose temperature measurement time is similar to a temperature measurement time of the first user.

When the body temperature management server 1 does not receive a measured body temperature from the first user terminal 31 of the first user and the second user being a manager of a group to which the first user belongs confirms body temperature information about a certain member in the group, confirmation of the body temperature information about the member by the second user may be notified to the first user terminal 31.

The body temperature management server 1 may notify the first user terminal 31 of an optimal temperature measurement place for the first user and/or an optimal temperature measurement time for the first user. For instance, the body temperature management server 1 may detect an action pattern of the first user and give notification of a place where the first user stays longest in a period from waking up to going out as the optimal temperature measurement place for the first user. For instance, the body temperature management server 1 may give notification of a time period during which the first user stays in the optimal temperature measurement place for the first user as the optimal temperature measurement time for the first user.

The body temperature management server 1 may transmit alert information to the first user terminal 31 when body temperature information indicating a predetermined value or higher is continuously measured throughout a predetermined period. The predetermined value is, for example, 37.5° C., and the predetermined period includes, for example, four days.

The body temperature management server 1 may determine whether the first user has a fever, and transmit, to the first user terminal 31, a message recommending self-quarantine when the user has a fever. The body temperature management server 1 may determine that the first user has a fever when the body temperature information about the first user indicates 37° C. or higher.

The body temperature management server 1 may store schedule information about a plurality of users working at the same company. When body temperature information about one user indicates 37° C. or higher among the body temperature information about the users, the body temperature management server 1 may transmit alert information to a user terminal of each user, other than the one user, who is to report to work.

The body temperature management server 1 may change a frequency of giving notification of a message encouraging measurement of a body temperature to the first user in accordance with implementation or non-implementation of a preventive measure against a virus spread (e.g., issuance or non-issuance of a declaration of a state of emergency from a government). Specifically, the body temperature management server 1 may set a frequency of notification of a message encouraging measurement of a body temperature to the first user in implementation of a preventive measure against a virus spread to be higher than a frequency of the notification of the message encouraging measurement of a body temperature to the first user in non-implementation of the preventive measure against the virus spread.

The body temperature management server 1 may change wording in a message encouraging measurement of a body temperature for the notification to the first user who has not measured a body temperature thereof in accordance with implementation or non-implementation of a preventive measure against a virus spread (e.g., issuance or non-issuance of a declaration of a state of emergency from a government). Specifically, the body temperature management server 1 may notify the first user of a message saying, “Make sure to measure your body temperature.”, in implementation of a preventive measure against a virus spread, and may notify the first user of a message saying, “Measure your body temperature.”, in non-implementation of the preventive measure against the virus spread.

The body temperature management system may further include a Web meeting server communicably connecting a plurality of user terminals to one another via a network to hold a Web meeting by sharing images or pictures and voices acquired at the respective terminals. Each user terminal may receive an input of a body temperature of each user and a current health condition of each user, and transmit the input body temperature information and health condition information to the body temperature management server 1. The body temperature management server 1 may transmit the body temperature information and the health condition information received from each user terminal to the Web meeting server. When the Web meeting is held among a plurality of users, the Web meeting server may display, on a Web meeting screen image, an icon indicating a body temperature having a predetermined value or higher about a certain user or an icon indicating a symptom of a specific disease from a health condition of the user together with an image of the user. For instance, when the user has the body temperature of 37° C. or higher, the Web meeting server may display, on the Web meeting screen image, an icon indicating that the user has a fever together with an image of the user. Besides, for instance, when the health condition of the user indicates coughing, the Web meeting server may display, on the Web meeting screen image, an icon indicating the coughing of the user together with an image of the user. In this manner, each user can know a body temperature and a health condition of other user in the Web meeting.

In addition, the first user terminal 31 may receive an input of a residence of the first user and a current health condition of the first user, and transmit the input residence information and health condition information to the body temperature management server 1. When the health condition information about the first user shows a symptom of a specific disease, the body temperature management server 1 may specify a hospital or clinic suitable for treatment of the specific disease, and notify the first user terminal 31 of the specified hospital or clinic.

In each embodiment, each constituent element may be realized with dedicated hardware or by executing a software program suitable for the constituent element. Each constituent element may be realized by a program execution unit, such as a CPU or a processor, reading out and executing a software program recorded on a recording medium, such as a hard disk or a semiconductor memory. Other independent computer system may implement a program by recording the program in a recording medium to be transferred, or transferring the program via a network.

A part or all of functions of the device according to the embodiment of the disclosure are typically realized as a large scale integration (LSI), which is an integrated circuit. These functions may be formed as separate chips, or some or all of the functions may be included in one chip. The circuit integration is not limited to the LSI, and may be realized with a dedicated circuit or a general-purpose processor. A field programmable gate array (FPGA) that is programmable after manufacturing of an LSI or a reconfigurable processor in which connections and settings of circuit cells within the LSI are reconfigurable may be used.

A part or all of functions of the device according to the embodiment of the present disclosure may be implemented by a processor, such as a CPU executing a program.

Numerical values used above are merely illustrative to be used to specifically describe the present disclosure, and thus the present disclosure is not limited to the illustrative numerical values.

Order in which steps shown in the flowcharts are executed is merely illustrative to be used to specifically describe the present disclosure, and thus steps may be executed in order other than the above order as long as similar effects are obtained. Some of the steps may be executed simultaneously (in parallel) with other steps.

### INDUSTRIAL APPLICABILITY

The technology according to this disclosure achieves presentation of alert information in accordance with an environment around a user or a situation of the user, and therefore is useful as a technology of acquiring body temperature information indicating a body temperature of the user and presenting the acquired body temperature information to another user.

1. An information processing method, by a computer, comprising:
acquiring body temperature information indicating a body temperature of a user and associated with a user ID specifying the user;
acquiring user information about the user associated with the user ID;
acquiring an alert criterion in accordance with the user information from an alert criteria database;
determining whether the body temperature information satisfies the alert criterion;
generating alert information for issuing an alert about a health state of the user in association with the user ID when the body temperature information is determined to satisfy the alert criterion; and
outputting the alert information.

2. The information processing method according to claim 1, further comprising acquiring dangerous level information indicating a dangerous level per region in accordance with a virus spread situation, wherein
the user information includes residence information indicating a residence of the user;
the alert criteria database associates a plurality of dangerous levels with alert criteria for the body temperature, and,
in the acquiring of the alert criterion, a specific dangerous level for the residence of the user is specified on the basis of the dangerous level information, and the alert criterion associated with the specified dangerous level is acquired from the alert criteria database.

3. The information processing method according to claim 1, wherein the user information includes the number of contact persons with the user on a previous day,
the alert criteria database associates the number of contact persons with the alert criterion for the body temperature, and,
in the acquiring of the alert criterion, the alert criterion associated with the acquired number of contact persons with the user on the previous day is acquired from the alert criteria database.

4. The information processing method according to claim 1, further comprising outputting the body temperature information when the body temperature information is determined to dissatisfy the alert criterion.

5. The information processing method according to claim 1, wherein the alert criteria database further associates the alert criterion with an alert level,
the information processing method further comprising:
specifying the alert level associated with the alert criterion when the body temperature information is determined to satisfy the alert criterion; and
specifying, with reference to an alert destination database associating the alert level with an alert destination, the alert destination associated with the specified alert level, wherein,
in the outputting of the alert information, the alert information is output to the specified alert destination.

6. The information processing method according to claim 1, wherein the alert criteria database further associates the alert criterion with an alert level,
the information processing method further comprising:
specifying the alert level associated with the alert criterion when the body temperature information is determined to satisfy the alert criterion, wherein
in the generating of the alert information, the alert information having a display manner in accordance with the specified alert level is generated.

7. The information processing method according to claim 1, wherein the user information includes at least one of: health condition information indicating a health condition of the user; a past body temperature history of the user: residence information indicating a residence of the user; underlying disease information about an underlying disease of the user; age information indicating the age of the user; and contact person information indicating the number of contact persons with the user on a previous day,
the alert criteria database includes a first alert criterion for the body temperature, and a second alert criterion for at least one of: a health condition; a fever continuous day number indicating the number of days when the body temperature continuously shows a predetermined temperature or higher; a dangerous level per region in accordance with a virus spread situation; the underlying disease; the age; and the number of contact persons on the previous day,
in the determining whether to satisfy the alert criterion, the body temperature information is determined whether to satisfy the first alert criterion, and at least one of the health condition information, the fever continuous day number, a dangerous level in the residence, the underlying disease information, the age information, and the contact person information is determined whether to satisfy the second alert criterion, and
the alert information is generated when the body temperature information is determined to satisfy the first alert criterion, and the at least one of the health condition information, the fever continuous day number, the dangerous level in the residence, the underlying disease information, the age information, and the contact person information is determined to satisfy the second alert criterion.

8. An information processing device, comprising:
a body temperature information acquisition part that acquires body temperature information indicating a body temperature of a user and associated with a user 1D specifying the user;
a user information acquisition part that acquires user information about the user associated with the user ID;
an alert criterion acquisition part that acquires an alert criterion in accordance with the user information from an alert criteria database;
a determination part that determines whether the body temperature information satisfies the alert criterion;
a generation part that generates alert information for issuing an alert about a health state of the user in association with the user ID when the body temperature information is determined to satisfy the alert criterion; and
an output part that outputs the alert information.

9. A non-transitory computer readable recording medium storing an information processing program, comprising:
causing a computer to execute:
acquiring body temperature information indicating a body temperature of a user and associated with a user ID specifying the user;
acquiring user information about the user associated with the user ID;
acquiring an alert criterion in accordance with the user information from an alert criteria database;
determining whether the body temperature information satisfies the alert criterion;
generating alert information for issuing an alert about a health state of the user in association with the user ID when the body temperature information is determined to satisfy the alert criterion; and
outputting the alert information.
